(19) United States
(12) Patent Application Publication
(10) Pub. No.: US 2005/0113693 A1
(43) Pub. Date: May 26, 2005

(54) Kits including 3-D ultrasound imaging catheters, connectable deployable tools, and deployment devices for use in deployment of such tools

(76) Inventors: Stephen W. Smith, Durham, NC (US); Warren Lee, Clifton Park, NY (US); J. Fritz Angle, Charlottesville, VA (US); Edward D. Light, Durham, NC (US)

Correspondence Address: MYERS BIGEL SIBLEY & SAJOVEC, PO BOX 37428, RALEIGH, NC, 27627, US

(21) Appl. No.: 10/958,046

(22) Filed: Oct. 4, 2004

Related U.S. Application Data
(60) Provisional application No. 60/508,542, filed on Oct. 3, 2003.

(51) Int. Cl. A61B 8/00; A61B 17/00

(52) U.S. Cl. 600/439; 600/459

(57) ABSTRACT

A kit for use in ultrasound imaging can include a deployment device configured for partial insertion in vivo, a 3-D imaging catheter, moveably coupled to the deployment device including a 2D ultrasound transducer phased array mounted thereon and configured to provide 3-D images, and a deployable tool coupled to the 3-D imaging catheter and configured to move in vivo in response to guidance thereof via the deployment device using the 3-D images.

### CROSS-REFERENCE TO RELATED PROVISIONAL APPLICATION

This application claims the benefit of Provisional Application Ser. No. 60/508,542, entitled 3-D ultrasound imaging catheters for use in deployment of vascular aneurysm grafts and methods of deployment of same, filed on Oct. 3, 2003, assigned to the assignee of the present invention, the disclosure of which is hereby incorporated herein by reference in its entirety as if set forth fully herein.

### STATEMENT OF GOVERNMENT SUPPORT

This invention was made with Government support under grant number HL64962 from the National Institutes of Health. The Government has certain rights to this invention.

### FIELD OF THE INVENTION

This invention relates generally to the field of imaging, and more particularly to the field of ultrasound imaging.

### BACKGROUND

It is known to implant endovascular grafts to repair thoracic and/or abdominal aortic aneurysms. To assist in such implants, a spiral Computerize Tomographic scan (CT) can be performed, with or without a contrast agent, to measure the dimensions of the area in which the implant is to be performed to determine proper sizing of the graft. Furthermore, fluoroscopy can be used during the implant, with or without a contrast agent, so that the graft is positioned properly. Use of these imaging techniques (i.e. CT and fluoroscopy) can result in undesirably large radiation exposure to both the patient and the medical personnel associated with the implant. Furthermore, possible side effects associated with the use of the contrast agent may occur.

### SUMMARY

Embodiments according to the invention can provide kits including 3-d ultrasound imaging catheters, connectable deployable tools, and deployment devices for use in deployment of such tools. Pursuant to these embodiments, a kit for use in ultrasound imaging can include a deployment device configured for partial insertion in vivo, a 3-D imaging catheter, moveably coupled to the deployment device including a 2D ultrasound transducer phased array mounted thereon and configured to provide 3-D images, and a deployable tool coupled to the 3-D imaging catheter and configured to move in vivo in response to guidance thereof via the deployment device using the 3-D images.

In some embodiments according to the invention, the deployable tool is a vascular graft. In some embodiments according to the invention, the deployable tool is a retriever including a coiled portion configured to capture an obstruction in vivo. In some embodiments according to the invention, the deployable tool is offset from the 3-D imaging catheter. In some embodiments according to the invention, the deployable tool surrounds the 3-D imaging catheter.

In some embodiments according to the invention, the retriever is configured for use to treat brain clots. In some embodiments according to the invention, the deployable tool is a retriever including a coiled portion configured to capture an obstruction in vivo. In some embodiments according to the invention, the deployable tool is a Guglielmi coil including a coiled portion. In some embodiments according to the invention, the Guglielmi coil is configured for use to treat cerebral aneuysms. In some embodiments according to the invention, the Guglielmi coil is offset from the 3-D imaging catheter. In some embodiments according to the invention, the Guglielmi coil surrounds the 3-D imaging catheter.

In some embodiments according to the invention, the deployable tool is a Left Atrial Appendage occlusion device configured to expand in vivo. In some embodiments according to the invention, the Left Atrial Appendage occlusion device is offset from 3-D imaging catheter.

### BRIEF DESCRIPTION OF THE DRAWINGS

FIG. 1 is a schematic illustration of the deployment device that can be included in a kit along with other components according to some embodiments of the invention.

FIG. 2 is a schematic diagram that illustrates embodiments of grafts having catheters with an array of ultrasound transducers thereon to provide 3-D forward scans of a portion of the graft and a portion of an area in which the graft is to be implanted.

FIG. 3 is a schematic diagram that illustrates embodiments of grafts with catheters including an array of ultrasound transducers that provide 3-D rear scans of a portion of the graft and a portion of an area in which the graft is to be implanted.

FIG. 4 is a schematic illustration of a deployable tool configured for use with a 3-D imaging catheter according to some embodiments of the invention.

FIG. 5 is a schematic illustration of a deployable tool configured for use with a 3-D imaging catheter according to some embodiment of the invention.

FIGS. 6A-6D are schematic illustrations of a Left Atrial Appendage occlusion device filter at various stages of deployment according to some embodiment of the invention.

FIG. 7 is a photograph of an IVC filter with a 3D imaging catheter and a capture device according to some embodiments of the invention.

### DESCRIPTION OF EMBODIMENTS ACCORDING TO THE INVENTION

The present invention now will be described more fully hereinafter with reference to the accompanying figures, in which embodiments of the invention are shown. This invention may, however, be embodied in many alternate forms and should not be construed as limited to the embodiments set forth herein. Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention as defined by the claims. Like numbers refer to like elements throughout the description of the figures.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It will be understood that, although the terms first, second, etc. may be used herein to describe various elements, these elements should not be limited by these terms. These terms are only used to distinguish one element from another. For example, a first portion could be termed a second portion, and, similarly, a second portion could be termed a first portion without departing from the teachings of the disclosure.

Spatially relative terms, such as “above”, “below”, “upper”, “lower”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly. Well-known functions or constructions may not be described in detail for brevity and/or clarity.

Embodiments of the invention are described herein with reference to schematic illustrations of idealized embodiments of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Thus, the elements illustrated in the figures are schematic in nature and their shapes are not intended to illustrate the actual shape of a region of a device and are not intended to limit the scope of the invention.

Embodiments according to the invention can provide kits including a deployable device 52 with an associated catheter 60 having a two dimensional array of transducers thereon to provide 3-D intraluminal scan (i.e., a 3-D imaging catheter), as well as an associated deployable tool 55 associated with the 3-D imaging catheter 60. In some embodiments according to the invention, the associated deployable tool 55 is configured to be deployed using the deployment device 52, which can be controlled responsive to the images provided by the 3-D imaging catheter 60. For example, in some embodiments according to the invention, the parts of the kit can be assembled so that the 3-D imaging catheter 60 and the associated deployable tool 55 are manipulated inside a patient using the images provided by the 3-D imaging catheter 60. Once the user is satisfied with the placement of the deployable tool 55, the deployable tool 55 can be deployed using the deployment device 52. In some embodiments according to the invention, the associated deployable tool 55 can be a vascular graph suitable for deployment to treat a variety of aneurysms, such as aortic aneurysms or cerebral aneurysms. In still other embodiments according to the invention, the deployable tool 55 can be what is commonly referred to as a “retriever,” used to remove obstructions, such as a blood clot, from inside a patient. In still other embodiments according to the invention, the deployable tool 55 may be a Left Atrial Appendage (LAA) occlusion device used to treat thrombosis in patients having a risk of atrial fibrillation.

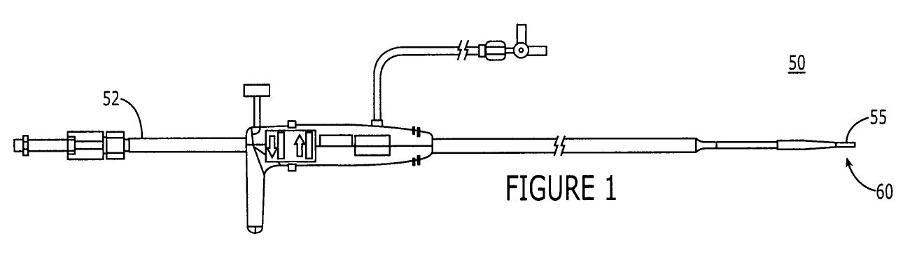

FIG. 1 is a schematic illustration of a kit including several components according to some embodiments of the invention. According to FIG. 1, the deployment device 52 can be configured to allow the insertion of the 3-D imaging catheter 60 along with an associated deployable tool 55 for guidance in deployment within a patient. For example, some embodiments according to the invention can provide catheters have two dimensional arrays of transducers thereon to provide 3-D intraluminal scans that can improve the visualization of an intraluminal region to a viewer. Ultrasound transducer arrays incorporated into intraluminal catheters are disclosed, for example, in U.S. Pat. Nos. 6,066,096 and in U.S. Pat. No. 6,530,888, which is included herewith. The catheters can be included in kits for use with vascular grafts to thereby improve the guidance, sizing, and deployment of grafts in interior regions such as in the deployment of abdominal aortic aneurysm vascular grafts.

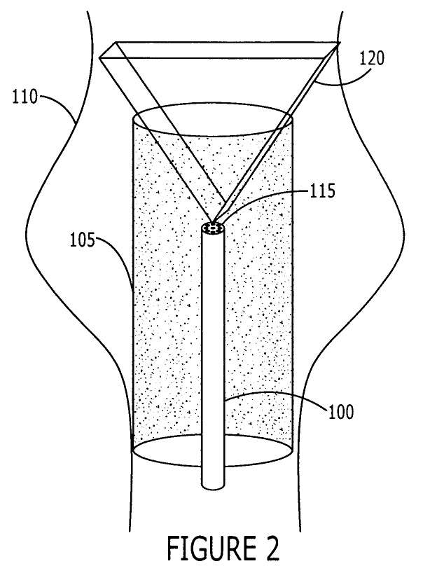

In some embodiments according to the invention, as shown for example in FIG. 2, a catheter 100 can be positioned along an axis of a graft 105 (or deployable tool) that is configured to be deployed proximate to an aneurysm 110. A two dimensional ultrasound transducer array 115 is located on a portion of the catheter 100 (i.e., a 3D imaging catheter) that is proximate to the graft 105 and is configured to provide forward-looking 3-D pyramidal scans of a region into which the catheter 100 and graft 105 are inserted. The forward scanning of the ultrasound transducer array 115 can provide a 3-D pyramidal scan 120 of a region proximate to the aneurysm 110 which can assist a user in guiding and deploying the graft 105 in the proper position relative to the aneurysm 110 using the deployment device 52. For example, the pyramidal scan 120 may provide the user with better visualization as to the location of the aneurysm 110 relative to the graft 105 and may thereby allow the user to place the graft 105 in an effective position for deployment relative to the aneurysm 110 and may also enable the user to make a more accurate estimate of the proper sizing of the graft.

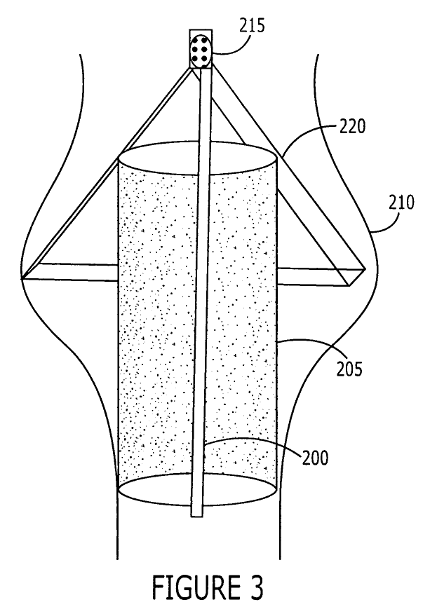

In some embodiments according to the invention, as shown for example in FIG. 3, a catheter 200 (i.e., a 3D imaging catheter) is located along an axis of a graft (or deployable tool) 205. A two dimensional ultrasound transducer array 215 is located on a portion of the catheter 200 that is proximate to the graft 205 and is configured to provide rear-looking 3-D pyramidal scans of a region into which the catheter 200 and graft 205 are inserted. The rear-looking scanning of the ultrasound transducer array 215 can provide a 3-D pyramidal scan 220 of a region proximate to the aneurysm 210 which can assist a user in guiding and deploying the graft 205 in the proper position relative to the aneurysm 210 using the deployment device 52. For example, the pyramidal scan 220 may provide the user with better visualization as to the location of the aneurysm 210 relative to the graft 205 and may thereby allow the user to place the graft 205 in an effective position for deployment relative to the aneurysm 210. Although the embodiments disclosed above are described as having forward and/or rear-looking scanning, it will be understood that other scanning directions may also be use in embodiments according to the invention.

It will be understood that embodiments according to the invention can also be used to provide deployment of venous filters in treating deep vein thrombosis. In some embodiments according to the invention, the deployable tool can be an inferior vena cava (IVC) filter as illustrated, for example, in FIG. 7. According to FIG. 7, the IVC (F) is shown with a 14 French 3D imaging catheter and a capture device (C).

It will also be understood that embodiments according to the invention can include rear-looking and forward-looking ultrasound transducer arrays. It will be further understood that catheters according to the invention can be included in kits used to deploy grafts of the type(s) discussed in U.S. Pat. No. 4,617,932 to Kornberg and in U.S. Pat. No. 5,522,883 to Slater et al., which are included herewith. It will be further understood that embodiments according to the invention can be used in other applications.

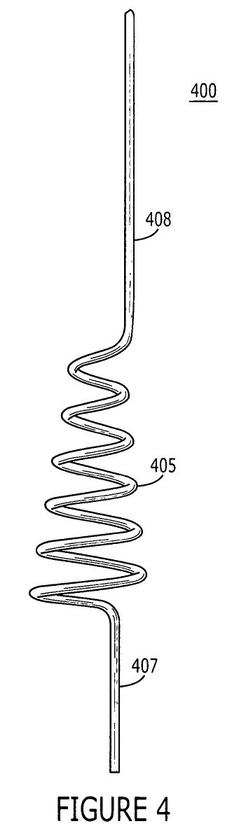

FIG. 4 is a schematic illustration of a deployable tool configured for use with a 3-D imaging catheter according to some embodiments of the invention. In particular, a retriever 400 shown in FIG. 4, can be movably coupled to the 3-D imaging catheter according to embodiment of the invention, so that the retriever 400 can be retracted so that it is effectively removed from the field of imaging provided by the 3-D imaging catheter. The retriever 400 is further configured to be deployed forward using the deployment device to capture an occlusion (and ultimately removed from) inside the patient, such as a brain clot. As will be understood by those skilled in the art, the retriever 400 may be rotated to “corkscrew” into the occlusion so that it can be removed when the catheter and the retriever 400 are retracted from inside the patient using the deployment device. In some embodiments according to the invention, the retriever 400 is configured to surround the 3-D imaging catheter so that the 3-D imaging catheter passes through the coils 405 of the retriever 400. In other embodiments according to the invention, the retriever 400 is offset to one side of the 3-D imaging catheter. In still other embodiments according to the invention, the retriever 400 is retracted (prior to deployment) so that the coils 405 are elongated to substantially resemble the first and second linear portions 408 and 407 of the retriever 400. When the retriever 400 is deployed, the coils 405 of the retriever 400 take shape as they may not longer be restrained, for example, by a jacket that encloses the coils when the retriever 400 is retracted. It will be understood that the deployable tool can be a Guglielmi detachable coil of the type that is typically used to treat cerebral aneurisyms according to other embodiments of the invention. Examples of Guglielmi detachable coils are illustrated, for example, on the world wide web at “radiologyinfo.org”

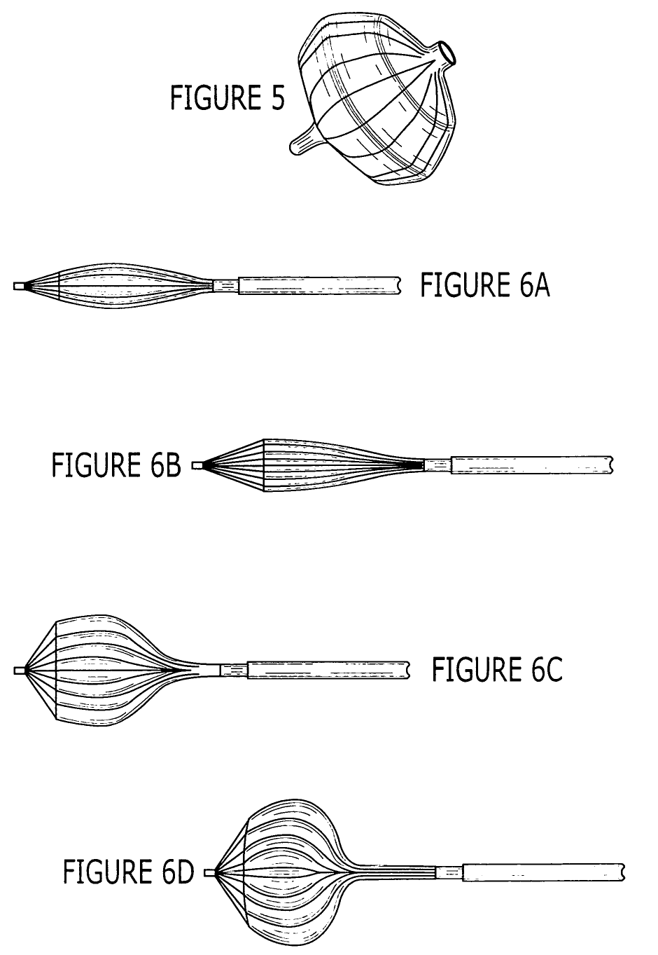

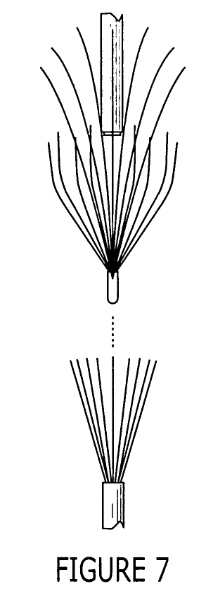

FIG. 5 is a schematic illustration of a Left Atrial Appendage (LAA) occlusion device deployable tool configured for use with a 3-D imaging catheter according to some embodiments of the invention. The LAA occlusion device is offset from the 3-D imaging catheter so that it is outside the imaging field of the 3-D imaging catheter when retracted. When the LAA occlusion device is deployed, the LAA occlusion device moves forward into the imaging field of the 3-D imaging catheter and expands as illustrated in FIGS. 6A-D.

Many alterations and modifications may be made by those having ordinary skill in the art, given the benefit of present disclosure, without departing from the spirit and scope of the invention. Therefore, it must be understood that the illustrated embodiments have been set forth only for the purposes of example, and that it should not be taken as limiting the invention as defined by the following claims. The following claims are, therefore, to be read to include not only the combination of elements which are literally set forth but all equivalent elements for performing substantially the same function in substantially the same way to obtain substantially the same result. The claims are thus to be understood to include what is specifically illustrated and described above, what is conceptually equivalent, and also what incorporates the essential idea of the invention.

1. A kit for use in ultrasound imaging comprising:
a deployment device configured for partial insertion in vivo;
a 3-D imaging catheter, moveably coupled to the deployment device, including a 2D ultrasound transducer phased array mounted thereon and configured to provide 3-D images; and
a deployable tool coupled to the 3-D imaging catheter and configured to move in vivo in response to guidance thereof via the deployment device using the 3-D images.

2. A kit according to claim 1 wherein the deployable tool comprises a vascular graft.

3. A kit according to claim 1 wherein the deployable tool comprises a retriever including a coiled portion configured to capture an obstruction in vivo.

4. A kit according to claim 3 wherein the deployable tool is offset from the 3-D imaging catheter.

5. A kit according to claim 3 wherein the deployable tool surrounds the 3-D imaging catheter.

6. A kit according to claim 3 wherein the retriever is configured for use to treat brain clots.

7. A kit according to claim 1 wherein the deployable tool comprises a retriever including a coiled portion configured to capture an obstruction in vivo.

8. A kit according to claim 1 wherein the deployable tool comprises a Guglielmi coil including a coiled portion configured to capture an obstruction in vivo.

9. A kit according to claim 8 wherein the Guglielmi coil is configured for use to treat cerebral aneuysms.

10. A kit according to claim 8 wherein the Guglielmi coil is offset from the 3-D imaging catheter.

11. A kit according to claim 8 wherein the Guglielmi coil surrounds the 3-D imaging catheter.

12. A kit according to claim 1 wherein the deployable tool comprises a Left Atrial Appendage occlusion device configured to expand in vivo.

13. A kit according to claim 12 wherein the Left Atrial Appendage occlusion device is offset from 3-D imaging catheter.

14. A kit according to claim 1 wherein the deployable tool comprises a venous filter.
